(19) United States
(12) Patent Application Publication
(10) Pub. No.: US 2013/0231699 A1
(43) Pub. Date: Sep. 5, 2013

(54) LOCKING SUTURE

(71) Applicant: DR7 INNOVATIONS, LLC, Goshen, KY (US)

(72) Inventor: Edward Lyle Cain, JR., Goshen, KY (US)

(73) Assignee: DR7 INNOVATIONS, LLC, Goshen, KY (US)

(21) Appl. No.: 13/764,089

(22) Filed: Feb. 11, 2013

Related U.S. Application Data
(60) Provisional application No. 61/600,734, filed on Feb. 20, 2012.

(51) Int. Cl. A61B 17/04

(52) U.S. Cl. CPC A61B 17/0485
USPC 606/228

(57) ABSTRACT

A locking suture comprises a main suture branch with at least one member fixably attached, wherein the main suture branch and at least one member pass through a suture eyelet of the locking suture in a first direction, but passage of the member in a second direction through the suture eyelet is resisted due to mechanical impediment between the member and the eyelet.

### CROSS REFERENCE TO RELATED U.S. APPLICATION

This application claims priority to U.S. Provisional Application No. 61/600,734, filed on Feb. 20, 2012, the teachings and entire disclosure of which are fully incorporated herein by reference.

### BACKGROUND

Endoscopic surgery refers to surgical procedures performed through small incisions, or natural body openings. Various forms of endoscopic surgery include arthroscopy, laparoscopy, thoracoscopy, cystoscopy rhinoscopy, otoscopy, and microsurgery. Incisions associated with endoscopic surgery are generally small, and puncture-like. In some cases, one or more puncture wounds are made through skin and subcutaneous tissues using a trocar, which has a shaft with at least one pointed end and may be surrounded by a sleeve-like cannula. A surgeon then withdraws the trocar and inserts medical instruments through the cannula to conduct the surgery. The puncture-like incision(s) thus serve as an entry point to the surgical site. The size of the incision depends on the nature of the surgery, but may typically range from about 3 mm (millimeters) to about 10 mm.

Arthroscopy is one type of endoscopic surgery. In some kinds of arthroscopy, surgeons repair damaged or torn connective tissue (e.g., ligaments) by sewing one piece of tissue to another with sutures, allowing the tissue to heal without pulling apart. In other kinds, surgeons reattach tissue to surrounding bone using sutures, and, if indicated, additional devices, such as implantable anchors or other fixation members.

In some kinds of arthroscopy, one end of a suture is securely attached, or mounted, to a conventional surgical needle having a shank-like body member, which is formed from conventional materials, such as a metal or a metal alloy. A surgical needle typically contains a sharp point at one end for advancing into and through tissue, and a mounting apparatus at the opposite end. Various techniques for mounting a suture to a surgical needle are known and conventionally used, e.g., use of adhesives, heat shrinking, or simply threading a suture through an eyelet of the needle. For some uses, a suture and a surgical needle are combined as an unitary product.

Generally, sutures are formed from various materials as are known to persons of ordinary skill in the art, e.g., polyethylene, silk, catgut, chromic catgut, polyglycolic acid (PGA), polylactic acid (PLA), and polydioxanone (PDS), nylon, and polypropylene, which are mentioned here as non-limiting examples. Suture material sometimes is a monofilament material, while some sutures are formed from multi-stranded materials woven together, or braided. The core of a suture can be either hollow or solid. Some sutures are formed from bioabsorbable material, which is broken down over time and eventually eliminated from the body or absorbed by the tissues. Some sutures are formed from biocompatible material, meaning that a patient's body will generally tolerate the presence of the suture indefinitely. For some uses, a suture is coated with conventional materials to improve functional properties, such as durability, and the ease with which a suture passes through tissue as a function of reducing the friction at the tissue-suture interface.

In use, sufficient force is applied to a surgical needle to cause the sharp point to move through or around bodily tissue in a particular direction chosen by a surgeon, i.e., “antegrade” movement. The shank-like body member and attached suture follow the needle, which threads the suture through bodily tissue in a pattern determined by the surgeon. Once the pattern is complete, it is desirable for the suture to hold its position. This can be accomplished by establishing a friction force that resists or limits suture movement relative to tissue, thereby creating tension to hold the tissue in the desired position.

Forming knots in a suture or series of sutures is a traditional method for creating the friction force. However, for arthroscopy and other forms of endoscopic surgery performed through tiny openings, often the surgeon cannot visualize the tissue at the surgical site without the aid of cameras and similar viewing devices. Even if visualization is achieved, forming knots in such confined spaces is difficult, if not impractical.

Therefore, various knotless suturing techniques already exist. Some involve forming a slipknot or other closed loop at the trailing end of a suture, and then passing a portion (leading end) of the suture body through that loop. However, such techniques present various challenges and disadvantages, most notably the propensity of the suture body to undergo retrograde movement (i.e., in the opposite direction of antegrade) over time, causing it to loosen. This tends to reduce tension in the suture, allowing repaired tissue to pull apart over time. Therefore, it is desirable to limit such retrograde movement.

Whether securing ligament to bone or approximating two ends of a torn ligament, as well as other forms of tissue repair, as a surgical needle is advanced through the tissue, the mounted suture follows. In this way, both enter and exit tissue in alternating fashion according to the desired pattern. Either while the pattern is progressing, or once completed, the term “approximation” refers to the act of bringing and maintaining a section of tissue in close proximity to something else, usually either another section of tissue or a piece of bone. Once approximated, it is desirable to hold that position.

This is particularly true given the forces at work on the tissues of a person's body, including repaired tissue. In a soft tissue repair, the ends that are approximated and then securely attached will tend to be pulled away from each other, through physical motion (e.g., throwing a ball in a person who has undergone shoulder arthroscopy), gravity (e.g., a Bankart lesion that involves the inferior glenohumeral ligament), and blunt impact (e.g., falling and landing on the surgical site). Thus, it is desirable for a suture to be strong enough, and also held in place securely enough, that it will remain in a substantially static position as the suture counteracts such forces over time.

The use of bone anchors is also part of conventional practice in arthroscopy. These are used to help securely hold tissue to bone. A surgeon bores a small hole into a bone and threads an anchor into the hole. With the anchor serving as a secure fixation point, the surgeon then threads a suture through an aperture on the anchor.

### SUMMARY

A locking suture, as described and claimed herein, comprises at least a main branch, a leading end of which is of suitable size and dimension for passing through tissue when appropriate force in a desired direction is placed on the main branch, for example by attachment to a surgical needle that itself is passing through tissue; at least one suture locking braid fixably attached to the main branch, for resisting movement of the main branch in at least one direction; and an opening, positioned at or near a trailing end of the main branch, which may be formed integral to the main branch, for accommodating passage of a portion of the main branch and the at least one locking braid during antegrade movement of the main branch, but resisting retrograde passage of the at least one locking braid back through the suture opening. In some embodiments, the mechanical impediment is established by a physical constraint due to the size and dimension of suture eyelet 16 compared to the dimensions, size, length, and angle orientation of suture locking braid 18, wherein the mechanical stress associated with the impediment causes locking braid 18 to deform.

The main branch has a leading end and a trailing end. In use, e.g., during an endoscopic surgical procedure, the leading end is the end that passes through tissue before the trailing end does. The leading end also passes through the opening, which is a suture eyelet in some embodiments. The suture eyelet is of suitable size and dimension to receive the leading end and a first segment of the main branch, as well as at least one of the suture locking braids. The number of locking braids that pass through the suture eyelet influences the amount of tension applied to the suture and the surrounding tissue being repaired. This allows the suture to be securely used for repairing and reattaching tissue in various kinds of procedures, e.g., endoscopy, without having to manually tie knots. In general, tension is proportional to the number of suture locking braids that pass through the eyelet during antegrade movement. In some alternative embodiments, a plurality of outer members spaced along the main suture branch pass through the suture eyelet during antegrade movement, but limit retrograde passage back through the suture eyelet.

### BRIEF DESCRIPTION OF THE DRAWINGS

The drawings and embodiments described herein are illustrative of multiple alternative structures, aspects, and features of the present embodiments, and they are not to be understood as limiting the scope of present embodiments. It will be further understood that the drawing figures described and provided herein are not to scale, and that the embodiments are not limited to the precise arrangements and instrumentalities shown.

FIG. 1 is a plan view of a locking suture, according to multiple embodiments and alternatives.

FIG. 2 is a plan view of a locking suture, according to multiple embodiments and alternatives, with a region (depicted by circle A) illustrated in more detail in FIG. 3A.

FIG. 3A is a perspective view of the region of FIG. 2 indicated by the dotted circle, according to multiple embodiments and alternatives.

FIG. 3B is a perspective view of a locking suture, according to multiple embodiments and alternatives.

FIG. 4 depicts tissue repair using a locking suture, according to multiple embodiments and alternatives.

FIG. 5 depicts a surgical repair, according to multiple embodiments and alternatives, also utilizing a suture anchor (not claimed).

FIG. 6 is a perspective view of a locking suture, according to multiple embodiments and alternatives.

FIG. 7 is a cross-sectional view of an outer member around a locking suture main branch, along the section I-I from FIG. 6, according to multiple embodiments and alternatives.

FIG. 8 is perspective view of a locking suture, according to multiple embodiments and alternatives.

### MULTIPLE EMBODIMENTS AND ALTERNATIVES

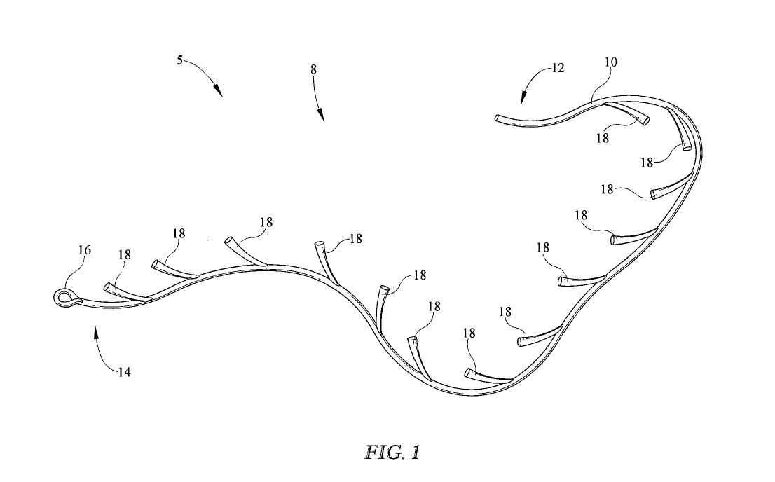

Multiple embodiments and alternatives of a locking suture are described and taught herein. As illustrated in FIG. 1, a locking suture 5 comprises a main branch 10 of suture strand 8, the suture strand and its main branch generally being formed from suture materials discussed above and through methods and techniques as are generally known in the art. In some embodiments, main branch 10 comprises an inner hollow core suture. Alternatively, main branch 10 has a solid core.

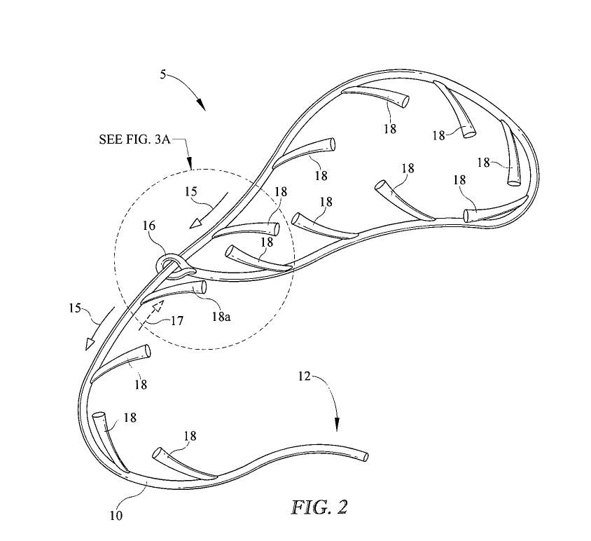

A first end of main branch 10 comprises leading end 12. An opposite end of main branch 10 comprises trailing end 14. In some embodiments, trailing end 14 includes suture eyelet 16, having an opening through which a first segment of suture strand 8 is passed, beginning with leading end 12. In FIG. 2, first segment is the main branch 10 and locking braids 18 (including 18a) shown between eyelet 16 and leading end 12. In some embodiments, suture eyelet 16 is formed from plastic or other solid materials.

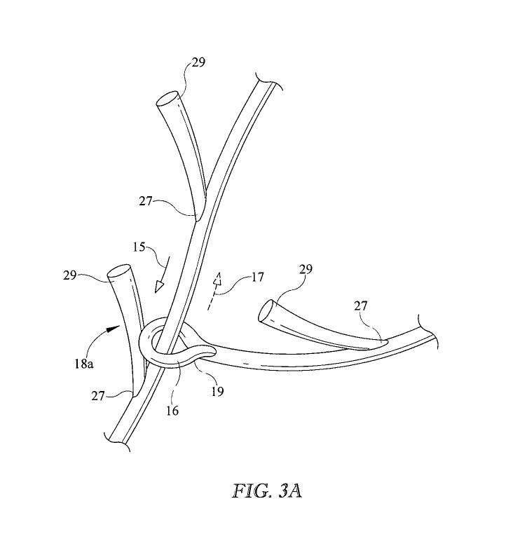

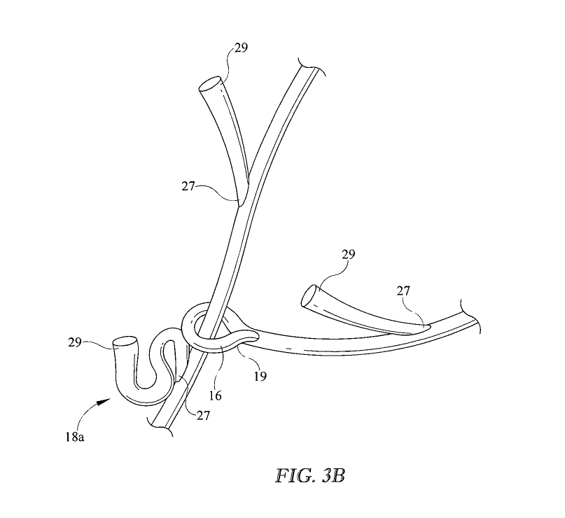

As also illustrated in FIGS. 1-5, in some embodiments, at least one suture locking braid 18 is attached to main branch 10. In some embodiments, each of a plurality of suture locking braids 18 is fixably attached to main branch 10 by securely joining a main branch connecting end 27 (see FIG. 3) of each suture locking braid 18 into the fibers of main branch 10. In some embodiments, this is accomplished by weaving main branch connecting end 27 of a suture locking braid 18 into the inner hollow core of main branch 10, through methods that are known to persons having ordinary skill in the art, sufficiently to prevent detachment of suture locking braid 18 from main branch 10 as both pass through suture eyelet 16 during antegrade movement. Alternatively, suture locking braid 18 is fixably attached to main branch 10 by other means, e.g., by thermally deforming (i.e., melting) or gluing end 27 of suture locking braid 18 to main branch 10, again which means are known to persons having ordinary skill in the art.

As illustrated in FIG. 3, in some embodiments, each suture locking braid 18 is securely joined to main branch 10 at an acute angle. This biases each suture locking braid 18 to pass through suture eyelet 16 during antegrade movement 15. It will be appreciated that, during retrograde movement, the acute angle formed by suture locking braid 18 and main branch 10 biases against passage through suture eyelet 16. Preferably, suture locking braid 18 and main branch 10 form an angle of about 30° to about 45°.

Thus, during retrograde movement i.e., movement in a second direction—depicted by arrow 17, when a suture locking braid 18 contacts suture eyelet 16 (i.e., a solid perimeter rim 19 at the outer edge of suture eyelet 16), braid 18 is mechanically impeded from passing through eyelet 16. The mechanical impediment is established by a physical constraint due to the size and dimension of suture eyelet 16 compared to the size, length, and angle orientation of suture locking braid 18. Upon sustained contact with the perimeter rim 19 of suture eyelet 16, suture locking braid 18 experiences a mechanical impediment such that, through sustained contact with rim 19, the suture locking braid becomes structurally deformed as seen in FIG. 3B. This structural deformation effectively increases the breadth of suture locking braid 18, including near its free end 29 (i.e., the end opposite main branch connecting end 27), and generally increases the surface area of suture locking braid 18 that contacts suture eyelet 16. The mechanical impediment described here thus limits (i.e., substantially prevents) retrograde passage of suture locking braid 18 back through the suture eyelet.

In some embodiments, suture locking braids 18 are generally formed from the same or similar materials as main branch 10. The spacing between suture locking braids 18 is generally variable. Spacing is influenced by various considerations, such as the diameter of main branch 10, the type of tissue being repaired, and the range of tension (force per unit length) required to retain the position of the suture over time. For example, if the level of tension required varies over a relatively wide range, then more distance in the spacing between suture locking braids 18 will generally be acceptable, assuming all other factors are equal. However, if a more precise amount of tension is needed, then reduced spacing between suture locking braids 18 is desirable. This generally increases the number of suture locking braids 18 along main branch 10, and increases the number of options for which suture locking braid will be the last one passed through suture eyelet 16.

For example, a #2 suture is used for relatively small tissue repairs. In some embodiments, a locking suture 8 of similar size and dimensions as a standard #2 suture has suture locking braids 18 positioned approximately 2-5 mm apart. By comparison, a #8 suture is meant for larger tissue repairs, and optionally has suture locking braids 18 positioned about 5 mm to about 8 mm apart. It is not necessary, however, that spacing between each of suture locking braids 18 should be must be limited to this range, nor is uniform spacing of them required along main branch 10. Rather, spacing of suture locking braids 18 is chosen according to multiple alternatives as selectably desired.

FIG. 2 illustrates a portion of main branch 10 beginning with leading end 12 having been passed through suture eyelet 16 for tissue (not shown in FIG. 2) repair. Antegrade movement arrow 15 depicts a first direction traveled by leading end 12, and retrograde movement arrow 17 (dashed arrow) depicts movement in a second direction generally opposite the first. Thus, locking suture 5, according to multiple embodiments and alternatives as described and taught herein, resists such retrograde movement due to the mechanical impediment created by the engagement of, and sustained contact between, a suture locking braid 18a with rim 19 of suture eyelet 16. The mechanical stress of this engagement causes locking braid 18a to deform. The change in shape of locking braid 18a through deformation (as shown in FIG. 3B) prevents its retrograde passage through eyelet 16, and this prevents further movement of the main branch 10 in the retrograde direction as well.

FIG. 3A illustrates a region as depicted by the dashed line circle shown in FIG. 2. A suture locking braid 18a is depicted as being positioned within this region, proximal to suture eyelet 16. The determination about which suture locking braid 18 is positioned proximal to suture eyelet 16 depends on the length of main branch 10 passed through suture eyelet 16. Further, while applicant's drawing figures are not to scale, FIG. 3A (among others) indicates the relative dimensions of main branch 10, suture locking braids 18, and suture eyelet 16. In some embodiments, if suture eyelet 16 is circular or substantially circular, then its diameter is slightly greater than the diameter of main branch 10 plus the diameter of each suture locking braid 18. These diameters are such that main branch 10 passes easily when it is passed through suture eyelet 16 by itself, but passage of main branch 10 combined with suture locking braid 18 makes for tighter passage.

As discussed herein, and shown in FIG. 3, each of suture locking braids 18 has a main branch connecting end 27 and a free end 29. In general, connecting end 27 is the part that fixably attaches to main branch 10, as described above. The dimensions of suture eyelet 16 generally permit main branch 10, and suture locking braid 18, to pass through its opening during antegrade movement (arrow 15). However, in response to retrograde movement (arrow 17) free end 29a does not pass retrograde back through suture eyelet 16, but rather deforms and is constrained against such retrograde movement upon engaging with perimeter 19 of suture eyelet 16.

By way of non-limiting example, the diameter of a #2-sized suture is about 0.5 mm. In some embodiments, a plurality of suture locking braids 18, each having a diameter of about 0.5 mm, are fixably attached to main branch 10 of a #2-sized suture. Given these dimensions, some embodiments would utilize a suture eyelet 16 having a diameter of about 0.5 mm to about 1.0 mm, and preferably of about 0.7 mm. During antegrade movement (arrow 15), dimensions on these orders allow relatively easy passage of main branch 10 by itself through suture eyelet 16, but passage of main branch 10 combined with a suture locking braid 18 is a tighter passage. In some embodiments, the length of each suture locking braid 18 is determined as a function of the diameter of suture eyelet 16. For example, in some embodiments, the length of each suture locking braid 18 is equal to or greater than about 2-5 times the difference between the diameter of suture eyelet 16 and main branch 10. Thus, it will be appreciated that surface area increases as diameter increases.

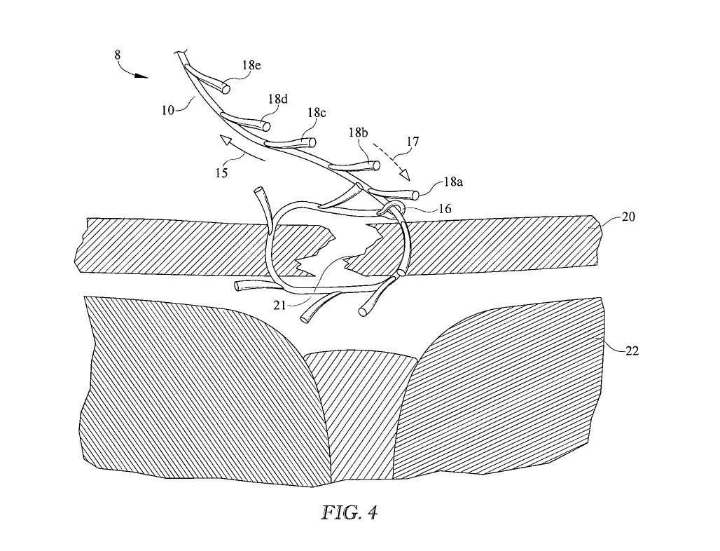

Present embodiments are used in multiple ways with various kinds of endoscopic surgeries, including arthroscopy. FIG. 4 illustrates a soft tissue repair using a suture 8, according to multiple embodiments and alternatives. Tissue 20 is torn at and along an area represented by tear 21. In various kinds of surgeries, e.g., arthroscopic, tissue 20 is proximal to bone 22. In the operation, a segment of main branch 10 is passed through one end of tissue 20 and then through another end of tissue 20, and this may be repeated multiple times according to a pattern as selectably chosen by a surgeon. When the pattern is complete, a surgeon passes a segment of main brain 10 having at least one suture locking braid 18 through suture eyelet 16. Direction of initial travel of main branch 10 is denoted by antegrade movement arrow 15. The length of the first segment of main branch 10 passing through suture eyelet 16 determines how many suture locking braids 18 also pass through. This can be as few as one suture locking braid. FIG. 4 shows suture locking braids 18a, 18b, 18c, 18d, and 18e as part of the first segment. However, it will be appreciated that the length of the first segment of main branch 10 passing through suture eyelet 16 is selectably chosen by a surgeon as a function of the tension desired to be placed on the tissue ends. Accordingly, retrograde movement depicted by arrow 17 is limited because the last suture locking braid 18a to pass through suture eyelet 16 cannot pass back to the other side. This is because, when suture locking braid 18a is engaged with sustained contact against suture eyelet 16, e.g., by manually pulling on the main branch in the retrograde direction, it deforms into a shape having dimensions larger than the dimension of the opening of suture eyelet 16.

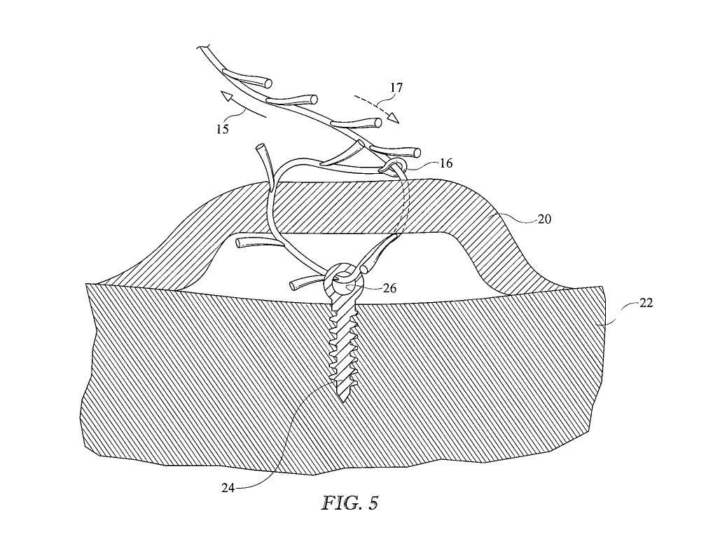

FIG. 5 further depicts a repair of tissue 20 that has partially torn away from bone 22. According to techniques and methods known in the art of surgical repair, suture anchor 24 having an anchor eyelet 26 may also be used as a fixation point for the repair. In this type of repair, main branch 10 is partially passed around a surface of tissue 20. It is passed through anchor eyelet 26 before completing at least one circuit around tissue 20. The dashed lines indicate main branch 10 passing “behind” tissue 20 (i.e., to the posterior side if tissue 20 is viewed anteriorly, and vice versa) before being passed through suture eyelet 16, all in a direction denoted by antegrade movement arrow 15. A very simple pattern is shown in FIG. 5, but it will be appreciated that a locking suture according to multiple embodiments and alternatives is used with many kinds of patterns, according to a surgeon's preference. Once main branch 10 and at least one suture locking braid 18 pass through suture eyelet 16, retrograde movement back through the suture eyelet (i.e., in a direction depicted by arrow 17) is limited as discussed above.

With respect to FIGS. 4 and 5, any items denoted by reference numerals 20-26 are not part of the claimed invention.

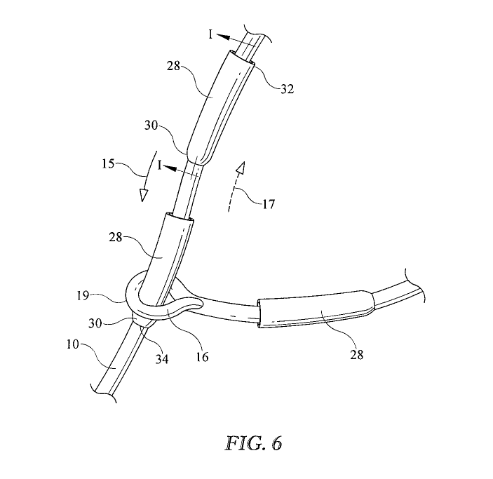

FIG. 6 illustrates an alternative embodiment of a locking suture 5, in which at least one outer member 28 (or a plurality of them, as is illustrated) substantially surrounds a portion of main branch 10. The purpose of outer members 28 is similar to that of suture locking braids 18, namely the limitation of and resistance to retrograde movement. Outer members 28 are generally formed from the same or similar materials as suture locking braids 18. Further, each outer member 28 has a nose 30 and a trailing edge 32. Generally, nose 30 has a forward-facing surface 34 oriented toward a first direction of travel, as indicated by directional arrow 15. In some embodiments, nose 30 is fixably attached to main branch 10 through conventional means known in the art, including but not limited to weaving nose 30 into the fibers of main branch 10, thermal deformation, and gluing nose 30 to main branch 10.

Generally, each outer member 28 is positioned such that, during antegrade movement (arrow 15), nose 30 travels through suture eyelet 16 before trailing end 32 does. In some embodiments, outer member 28 is an irregular cylinder, the circumference of which tapers from least at its nose 30 to greatest at trailing end 32. Alternatively, outer member 28 includes a beveled surface 31 such that the circumference at nose 30 is less than the circumference at trailing end 32. Accordingly, nose 30 passes through more easily than does trailing edge 32. Optionally, outer member 28 is formed from compressible material in order to facilitate passage through suture eyelet 16 during antegrade movement. In some embodiments, trailing edge 32 is sized, relative to the dimensions (e.g., diameter) of suture eyelet 16, so that it fits only snugly through suture eyelet 16 during antegrade movement (arrow 15), yet passage through suture eyelet 16 during antegrade movement is limited.

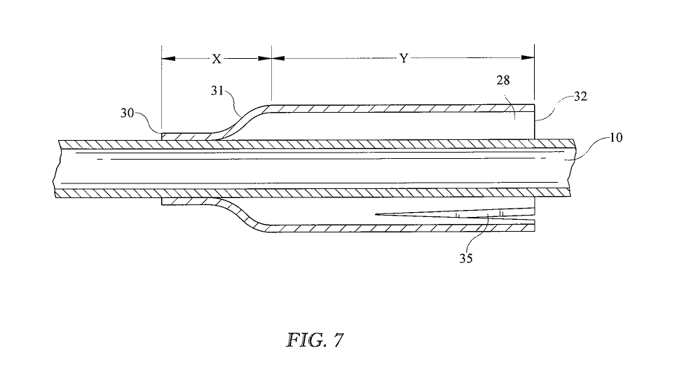

FIG. 7 is a cross-sectional view of one such outer member 28 surrounding main branch 10. Outer member 28 has a first region “X” that includes a beveled surface 31 flaring outward from main branch 10. A second region “Y” comprises a cylinder around main branch 10 terminating at trailing edge 32. In some embodiments, trailing edge 32 is a circular base, the circumference of which is greater than at any other portion of outer member 28.

In some alternative embodiments, the geometry of cylindrical member is substantially conical, wherein nose 30 is the most narrow portion of outer member 28, substantially resembling the vertex of a cone or truncated cone with a circular cross-section, the circular base of which is positioned at trailing edge 32. Optionally, the shape can be that of a partial cone or truncated cone with a semi-circular cross-section. Alternatively, outer member 28 is substantially pyramidal (not shown), wherein nose 30 is the most narrow portion of outer member 28, substantially resembling the vertex of a pyramid, the base of which is positioned at trailing edge 32. In some embodiments, the base, and therefore the cross-section of the pyramid, is chosen from the group triangular, square, rectangular, pentagonal, and hexagonal, with the number of lateral faces of the pyramid being equal to the number of sides forming the base.

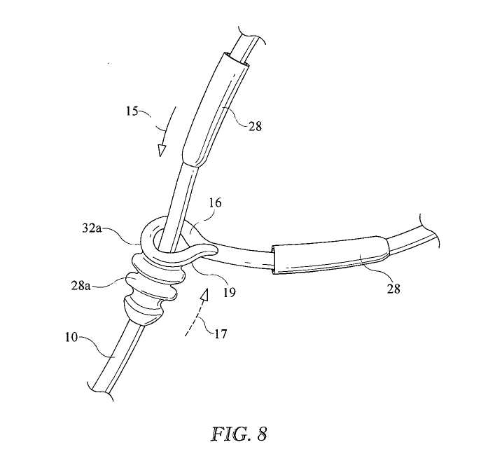

FIG. 8 shows a locking suture in use, with a plurality of outer members 28, including outer member 28a being the last one to pass through suture eyelet 16 in response to antegrade movement (arrow 15). Upon reaching that point, retrograde movement (arrow 17) of main branch 10 causes outer member 28a to contact perimeter 19 of suture eyelet 16. Upon sustained contact with perimeter 19 of suture eyelet 16, a mechanical impediment is created by the size and dimension of suture eyelet 16 compared to the size and width of outer member 28a. The mechanical impediment causes the latter to undergo buckling deformation, which may be marked by longitudinal shortening, as illustrated in FIG. 8. Deformation of outer member 28a increases its surface area, particularly of trailing edge 32a, relative to suture eyelet 16, in order to limit retrograde passage back through the suture eyelet. By limiting retrograde movement of main branch 10, suture strand 8 maintains a desired tension.

In some embodiments, each outer member 28 is fenestrated such that it further comprises at least one slit 35 running longitudinally along at least a portion of the surface of outer member 28. Slit 35 allows outer member 28 to compress as it passes through suture eyelet 16 during antegrade movement (arrow 15), yet the outer member fans out once passage is completed, thus increasing circumference at trailing edge 32. The increased circumference of trailing edge 32 further limits retrograde passage back through suture eyelet 16. In some embodiments, there are a plurality of slits 35. Preferably each outer member 28 has between two and four slits 35.

It will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways.

Also, it is to be understood that words and phrases used herein are for the purpose of description and should not be regarded as limiting. The use herein of “including,” “comprising,” “e.g.,” “containing,” or “having” and variations of those words is meant to encompass the items listed thereafter, and equivalents of those, as well as additional items.

Accordingly, the foregoing descriptions of several embodiments and alternatives are meant to illustrate, rather than to serve as limits on the scope of what has been disclosed herein. The descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

1. A locking suture, comprising:
a suture strand having a main branch with a first end and a second end and extending generally longitudinally;
an opening positioned at the second end of the main branch for accommodating passage of a first segment of the main branch that includes the first end when the first segment is passed through the opening while moving in a first direction of travel; and
at least one suture locking braid, at least a portion of which is fixably attached to the main branch;
wherein the first end is capable of being a leading end in the first direction of travel, and
wherein the at least one suture locking braid is configured to pass through the opening in the first direction of travel, and is further configured to resist passage through the opening in an opposite direction of travel to the first direction.

2. The locking suture of claim 1, wherein the main branch comprises a plurality of fibers surrounding a hollow inner portion.

3. The locking suture of claim 2, wherein the at least one suture locking braid is fixably attached to the main branch by weaving at least a portion of the suture locking braid into the plurality of fibers, wherein part of the suture locking braid occupies space within the hollow inner portion.

4. The locking suture of claim 2, wherein a portion of the at least one suture locking braid is not fixably attached to the main branch.

5. The locking suture of claim 1, wherein the at least one suture locking braid is positioned at an acute angle to the main branch.

6. The locking suture of claim 5, wherein the at least one suture locking braid is positioned at an angle of about 30° to about 45° to the main branch.

7. The locking suture of claim 1, wherein the first end and the second end are at opposite ends of the main branch.

8. The locking suture of claim 1, wherein the opening is formed integral to the main branch, and the shape of the opening is chosen from the group circular, semi-circular, rectangular, pentagonal, hexagonal, and octagonal.

9. The locking suture of claim 1, wherein the opening comprises an eyelet comprising a solid rim.

10. The locking suture of claim 1, wherein the at least one suture locking braid resists passage through the opening due to a mechanical impediment associated with the dimensions of the suture locking braid relative to the opening.

11. The locking suture of claim 10, wherein the mechanical impediment produces a structural deformation in the at least one suture locking braid.

12. A locking suture, comprising:
a suture strand having a main branch having a first end and a second end and extending generally longitudinally, wherein the first end of the main branch is capable of being a leading end in a first direction of travel of the suture strand;
an opening positioned at the second end of the main branch for accommodating passage of a first segment of the main branch that includes the first end when the first segment is passed through the opening while moving in a first direction of travel; and
at least one outer member fixably attached to the main branch, extending generally longitudinally and axially to the main branch;
wherein the at least one outer member is configured to pass through the opening in the first direction of travel, and further configured to resist passage through the opening in an opposite direction of travel to the first direction.

13. The locking suture of claim 12, wherein the at least one outer member comprises a first end comprising a nose having a forward-facing surface toward a first direction of travel, and a second end comprising a trailing edge facing a second direction of travel, wherein the surface area of the trailing edge exceeds that of the forward surface of the nose.

14. The locking suture of claim 13, wherein the cross-section of the at least one outer member is chosen from the group circular, semi-circular, triangular, square, rectangular, pentagonal, and hexagonal.

15. The locking suture of claim 13, wherein the main branch comprises a plurality of fibers surrounding a hollow inner portion.

16. The locking suture of claim 15, wherein the at least one outer member is fixably attached to the main branch by weaving a portion of the nose into the plurality of fibers.

17. The locking suture of claim 12, wherein the at least one outer member is fixably attached to the main branch by either thermal deformation or gluing a portion of the outer member to the main branch.

18. The locking suture of claim 12, wherein the surface of the at least one outer member is beveled, the two ends are circular, and the second end has a greater circumference than the first end.

19. The locking suture of claim 12, wherein the opening comprises an eyelet comprising a solid rim formed integral to the main branch.

20. The locking suture of claim 12, wherein the at least one outer member resists passage through the opening due to a mechanical impediment associated with the dimensions of the outer member relative to the opening.

21. The locking suture of claim 13, further comprising at least one slit in the trailing edge allowing for an enlargement of surface area of the trailing edge when a force is applied at the trailing edge.

22. The locking suture of claim 21, wherein the opening comprises an eyelet comprising a solid rim, and the applied force is associated with sustained contact between the trailing edge and the rim.

23. The locking suture of claim 22, wherein the at least one outer member is configured to undergo buckling deformation resulting in longitudinal shortening upon sustained contact between the trailing edge and the rim.

24. A locking suture for use with an anchor member, the anchor member having an opening for accommodating a first segment of the locking suture, comprising:
a suture strand having a main branch extending generally longitudinally, the main branch further including a first segment for passage through the opening of the anchor member; and
at least one member, at least a portion of which is fixably attached to the main branch;
wherein when the first segment passes through the opening in a first direction of travel, the at least one member is positioned to pass through the opening with the first segment, the at least one member being configured to resist passage through the opening in an opposite direction of travel to the first direction.

25. The assembly of claim 24, wherein the member is a suture locking braid or an outer member.
